(19) United States
(12) Patent Application Publication
(10) Pub. No.: US 2009/0198125 A1
(43) Pub. Date: Aug. 6, 2009

(54) OCT OPTICAL PROBE AND OPTICAL TOMOGRAPHY IMAGING APPARATUS

(75) Inventors: Koki NAKABAYASHI, Ashigarakami-gun (JP); Masahiro TOIDA, Ashigarakami-gun (JP); Kiichi KATO, Ashigarakami-gun (JP); Yutaka KOROGI, Ashigarakami-gun (JP); Tadashi MASUDA, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignees: FUJIFILM CORPORATION, Tokyo (JP); FUJINON CORPORATION, Saitama-shi (JP)

(21) Appl. No.: 12/363,021

(22) Filed: Jan. 30, 2009

(30) Foreign Application Priority Data
Feb. 1, 2008 (JP) 2008-022359
Jun. 24, 2008 (JP) 2008-164003

(51) Int. Cl. A61B 6/02; A61B 1/06; A61B 6/00

(52) U.S. Cl. 600/425; 600/160; 600/478

(57) ABSTRACT

An OCT optical probe to be inserted into a subject includes: a cylindrical sheath to be inserted into a subject; an optical fiber disposed in the internal space of the sheath; a rotatably-supporting portion fixed to the optical fiber in the vicinity of a distal end of the optical fiber; a distal optical system to deflect light emitted from the distal end of the optical fiber toward the subject; a holding portion to hold the distal optical system such that the optical system is rotatably supported by the rotatably-supporting portion; and a flexible shaft covering the optical fiber in the internal space, wherein the holding portion is fixed to a distal end of the flexible shaft. Using the OCT optical probe of the invention, the problem of degradation of measurement accuracy due to optical insertion loss and optical reflection loss at a rotary joint can be eliminated inexpensively and safely.

### BACKGROUND OF THE INVENTION

1. Field of the Invention

The present invention relates to an OCT optical probe and an optical tomography imaging apparatus, and particularly to an OCT optical probe having a function of scanning with light in a circumferential direction with respect to the long axis of the OCT optical probe, and an optical tomography imaging apparatus that acquires an optical tomographic image of a subject to be measured through OCT (Optical Coherence Tomography) measurement using the OCT optical probe.

2. Description of the Related Art

As a method for acquiring a tomographic image of a subject to be measured, such as a body tissue, a method using OCT measurement to acquire a tomographic image has been proposed. An OCT measurement system is one of optical interferometers. In the OCT measurement, low-coherent light emitted from a light source is divided into measurement light and reference light. The measurement light is applied to a subject to be measured, and then reflected light or backscattered light from the subject to be measured is combined with the reference light. Then, a tomographic image is acquired based on intensity of interference light formed between the reflected light and the reference light. Hereinafter, reflected light and backscattered light from the subject to be measured are collectively referred to as reflected light.

OCT measurement techniques are roughly classified into TD (Time Domain)-OCT measurement techniques and FD (Fourier Domain)-OCT measurement techniques.

In the TD-OCT measurement, the interference intensity is measured while the optical path length of the reference light is changed, thereby acquiring an intensity distribution of the reflected light corresponding to depth-wise positions in the subject to be measured.

In the FD-OCT measurement, the optical path lengths of the reference light and the signal light are fixed, and intensity of the interference light is measured for each spectral component of the light. Then, the thus acquired spectral interference intensity signals are subjected to frequency analysis, typically Fourier transformation, on a computer, thereby acquiring an intensity distribution of the reflected light corresponding to the depth-wise positions. Recently, the FD-OCT measurement is attracting attention since it does not require mechanical scanning on which the TD-OCT measurement relies, and therefore allows high speed measurement.

Typical systems that carry out the FD-OCT measurement include an SD (Spectral Domain)-OCT system and an SS (Swept Source)-OCT system.

The SD-OCT system uses wideband low-coherent light, decomposes the interference light into optical frequency components using a spectral means, measures intensity of the interference light for each optical frequency component using an arrayed photodetector, or the like, and applies Fourier transformation analysis to the thus acquired spectral interference waveform on a computer, to form a tomographic image.

The SS-OCT system uses, as a light source, a laser with optical frequency thereof swept with time, to measure temporal waveforms of signals corresponding to temporal changes of the optical frequency of the interference light, and applies Fourier transformation to the thus acquired spectral interference intensity signals on a computer, to form a tomographic image.

Further, it has been considered to combine any of the above-described optical tomography imaging systems with an endoscope for use in in-vivo measurement, and an OCT optical probe that can be inserted into a forceps channel of an endoscope has been known.

Such an OCT optical probe includes a distal end portion to be inserted in a body cavity, and a proximal end portion including a mechanism for moving light emitted from the distal end portion to scan in at least one-dimensional direction to acquire a tomographic image along a certain plane of the subject to be measured.

Japanese Patent No. 3104984 discloses an OCT optical probe that includes: a sheath to be inserted into a subject; a flexible shaft that is rotatable within the sheath about an axis extending in the longitudinal direction; an optical fiber covered with the flexible shaft; a distal optical system that deflects light emitted from the optical fiber at a substantially right angle with respect to the longitudinal direction, wherein the flexible shaft is rotated via a gear by a motor disposed at the proximal end, thereby rotating the distal optical system about the axis.

Jianping Su et al., “In vivo three-dimensional microelectromechanical endoscopic swept source optical coherence tomography”, Optics Express, Vol. 15, Issue 16, pp. 10390-10396, 2007, discloses, along with the development of MEMS (Micro Electro Mechanical Systems) techniques, an OCT optical probe that includes an MEMS motor disposed within the sheath in the vicinity of the distal end of the OCT optical probe, and a distal optical system fixed to the output shaft of the MEMS motor to rotate, so that the distal optical system is rotated about the shaft.

However, the conventional OCT optical probe disclosed in Japanese Patent No. 3104984, as shown in FIG. 15, includes a rotary joint disposed between the distal end portion inserted into a body cavity and the proximal end portion provided for moving the emitted light to scan. At the rotary joint, the optical fiber at the distal end portion side and the optical fiber at the proximal end portion side are optically coupled with the optical fibers being relatively rotated. Therefore, accuracy of the measurement may be degraded due to optical insertion loss and optical reflection loss caused, for example, by positional offset between optical axes of these fibers. Specifically, in a case where a commercially-available rotary joint is used, degradation of sensitivity due to the rotary joint is 10-20 dB.

In the OCT optical probe disclosed in Jianping Su et al., “In vivo three-dimensional microelectromechanical endoscopic swept source optical coherence tomography”, Optics Express, Vol. 15, Issue 16, pp. 10390-10396, 2007, as shown in FIG. 16, the light emitted from the distal end portion can be deflected to effect scanning without using a rotary joint. However, the MEMS motor is expensive and size reduction thereof is difficult, and therefore it may be difficult to insert the MEMS motor into the inner diameter of the forceps channel of the endoscope. Further, in order to prevent electrical shock to a human body, it is necessary to insulate a driving power supply to the MEMS motor at the distal end portion. In addition, a drive cable for the MEMS motor may block the light emitted from the distal end portion and affect image acquisition.

### SUMMARY OF THE INVENTION

In view of the above-described circumstances, the present invention is directed to providing an OCT optical probe and an optical tomography imaging apparatus using the OCT optical probe, that can inexpensively and safely eliminate the prior art problem of degradation in measurement accuracy due to optical insertion loss and optical reflection loss caused at optical coupling at a rotary joint disposed between an optical fiber at a distal end portion side and an optical fiber at a proximal end portion.

An OCT optical probe according to the invention includes: a substantially cylindrical sheath to be inserted into a subject, the sheath having an internal space; an optical fiber disposed in the internal space of the sheath along the longitudinal direction of the sheath; a rotatably-supporting portion integrally fixed to the optical fiber in the vicinity of a distal end of the optical fiber; a distal optical system to deflect light emitted from the distal end of the optical fiber toward the subject; a holding portion to hold the distal optical system such that the distal optical system is rotatably supported by the rotatably-supporting portion; and a flexible shaft covering the optical fiber in the internal space of the sheath, wherein the holding portion is fixed to a distal end of the flexible shaft. The term “substantially cylindrical” refers to a shape that may not necessarily be strictly cylindrical about a straight axis from one end to the other end, and the sheath may include a gently curved shape, such as a semispherical shape, at the distal end thereof. Further, the cross-sectional shape of the sheath may not necessarily be a mathematically-strict circle, and may be ellipsoidal, or the like. The “distal end” of the flexible shaft may not necessarily refer to the distal end of the flexible shaft, and may also refer to a position in the vicinity of the distal end.

The rotatably-supporting portion of the OCT optical probe according to the invention may include a bearing portion to rotatably support the holding portion.

Further, a fiber sheath to cover the optical fiber along the longitudinal direction may be provided between the optical fiber and the flexible shaft.

The distal end of the optical fiber of the OCT optical probe according to the invention may have an end face that is inclined by a predetermined angle with respect to a plane perpendicular to an optical axis of the optical fiber.

The OCT optical probe according to the invention may further include a cover glass, the proximal end of the cover glass may closely contact the distal end of the optical fiber, and the distal end of the cover glass may have a flat end face that is perpendicular to the optical axis.

The OCT optical probe according to the invention may further include a cover glass, the proximal end of the cover glass may closely contact the distal end of the optical fiber, and the distal end of the cover glass may have a convex end face that is adapted to collimate the light emitted from the distal end of the cover glass to be parallel to the optical axis.

An optical tomography imaging apparatus according to the invention is formed by an optical tomography imaging apparatus using any of the above-described measuring techniques, which employs the OCT optical probe according to the invention. Namely, the optical tomography imaging apparatus according to the invention includes: a light source unit to emit light; a light dividing unit to divide the light emitted from the light source unit into measurement light and reference light; an irradiation optical system to irradiate a subject to be measured with the measurement light; a combining unit to combine the reference light with reflected light of the measurement light reflected from the subject to be measured when the measurement light is applied to the subject; an interference light detecting unit to detect interference light formed between the combined reflected light and reference light; and a tomographic image processing unit to detect reflection intensity at a plurality of depth-wise positions in the subject to be measured based on frequency and intensity of the detected interference light, and to acquire a tomographic image of the subject to be measured based on the intensity of the reflected light at each of the depth-wise positions, wherein the irradiation optical system comprises the OCT optical probe of the invention.

### BRIEF DESCRIPTION OF THE DRAWINGS

FIG. 1 is a perspective view illustrating the entire portion of an optical tomography imaging apparatus, to which an OCT optical probe 1 of the invention is applied,

FIG. 2 illustrates a distal end portion 10 of the OCT optical probe 1 of the invention,

FIG. 3A illustrates a first embodiment of a bearing portion 17 of the OCT optical probe 1 of the invention,

FIG. 3B illustrates a second embodiment of the bearing portion 17 of the OCT optical probe 1 of the invention,

FIG. 4 illustrates the OCT optical probe 1 of the invention including a reflecting member,

FIGS. 5A and 5B illustrate the OCT optical probe 1 of the invention including a cover glass,

FIGS. 6A and 6B illustrate the OCT optical probe 1 of the invention including a cover glass with a convex distal end face,

FIG. 7 illustrates a proximal end portion 20 of the OCT optical probe 1 of the invention,

FIG. 8 illustrates pivot movement of the proximal end portion 20 of the OCT optical probe 1 of the invention,

FIG. 9 is a schematic structural diagram of an optical tomography imaging apparatus 100, to which the OCT optical probe 1 of the invention is applied,

FIG. 10 illustrates swept wavelength of light emitted from a light source unit 110,

FIGS. 11A and 11B illustrate a period clock signal generated by a period clock generating unit 120,

FIG. 12 is a schematic structural diagram of a tomographic image processing unit 150,

FIG. 13A illustrates an interference signal IS inputted to an interference signal acquiring unit 151,

FIG. 13B illustrates a rearranged interference signal IS,

FIG. 14 illustrates a tomographic image P generated by a tomographic information generating unit 154,

FIG. 15 is a schematic diagram illustrating a conventional OCT optical probe, and

FIG. 16 is a schematic diagram illustrating an OCT optical probe employing an MEMS motor.

### DESCRIPTION OF THE PREFERRED EMBODIMENTS

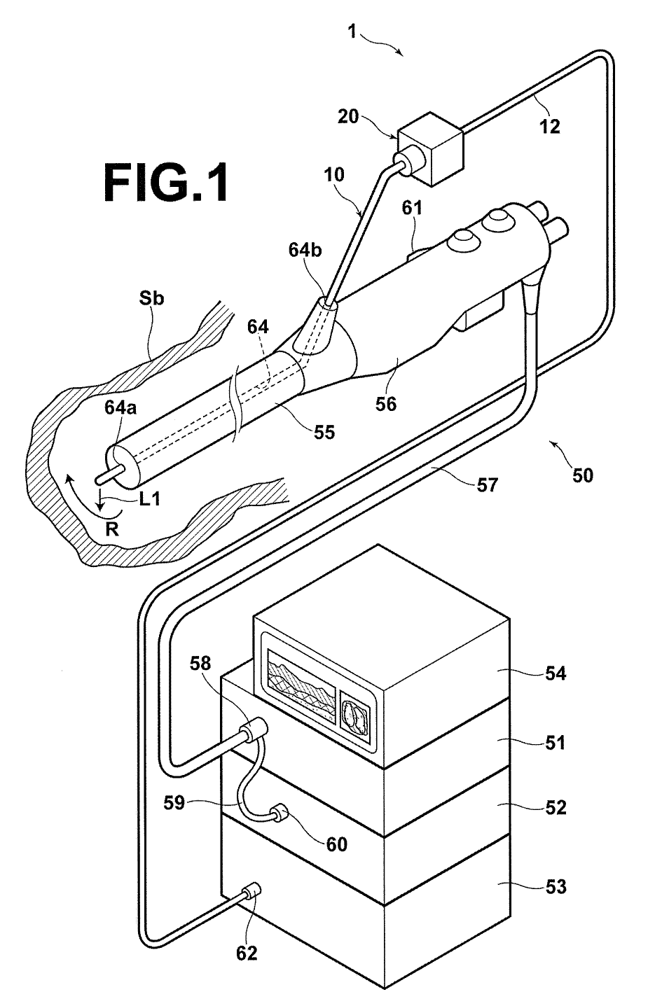

Hereinafter, embodiments of the present invention will be described with reference to the drawings. First, outline of an optical tomography imaging apparatus is described. FIG. 1 is a perspective view illustrating the entire portion of the optical tomography imaging apparatus, to which an OCT optical probe 1 of the invention is applied.

The optical tomography imaging apparatus includes: an endoscope 50 including the OCT optical probe 1; a light source unit 51, to which the endoscope 50 is connected; a video processor 52; an optical tomography processing unit 53; and a monitor 54 connected to the video processor 52.

The light source unit 51 applies measurement light L1 to a portion of a subject to be measured Sb, from which a tomographic image P is acquired, as described later.

The endoscope 50 includes a flexible and elongated insert portion 55, a manipulation unit 56 joined to the proximal end of the insert portion 55, and a universal code 57 extending from a side of the manipulation unit 56. A light source connector 58 is disposed at the end of the universal code 57, and the light source connector 58 is removably connected to the light source unit 51. A signal cable 59 extends from the light source connector 58, and a signal connector 60, which is removably connected to the video processor 52, is disposed at the end of the signal cable 59.

The insert portion 55 is inserted, for example, into a body cavity, and is used for observing the subject to be measured Sb. The distal end portion of the insert portion 55 is bendable, and a manipulation knob 61 for manipulating the distal end portion of the insert portion 55 to bend is provided at the manipulation unit 56. A forceps channel 64, which is a conduit shown by the dashed line in the drawing, is formed in the insert portion 55 along the longitudinal direction thereof, so that the OCT optical probe 1 or a treatment tool such as a forceps can be inserted through the forceps channel 64. One end of the forceps channel 64 is open at the distal end of the insert portion 55 to form a distal end opening 64a. The other end of the forceps channel 64 forms a forceps insertion port 64b, which is located above the manipulation unit 56. The OCT optical probe 1 is inserted through the forceps insertion port 64b and through the forceps channel 64, and the distal end of the OCT optical probe 1 is projected from the distal end opening 64a, so that the measurement light L1 can be applied to the subject to be measured Sb. It should be noted that, although not shown in the drawing, the distal end of the insert portion 55 is provided with an observation window used for observing the subject to be measured Sb, an illumination window through which the illumination light is applied, air and water supply nozzles used for removing dirt, and the like.

The OCT optical probe 1 includes a flexible and long distal end portion 10, a proximal end portion 20 joined to the proximal end of the distal end portion 10, and an optical fiber 12.

The distal end portion 10 is inserted through the forceps channel 64, which is shown by the dashed line in the drawing, to be inserted into a body cavity, as described above. The distal end portion 10 has a length of around 3 m.

One end of the optical fiber 12 is removably connected to the optical tomography processing unit 53 via an optical tomography connector 62, and the other end of the optical fiber 12 is inserted through the proximal end portion 20 and the distal end portion 10 to extend to an area in the vicinity of the distal end of the distal end portion 10.

Now, the OCT optical probe 1 of the invention is described in detail.

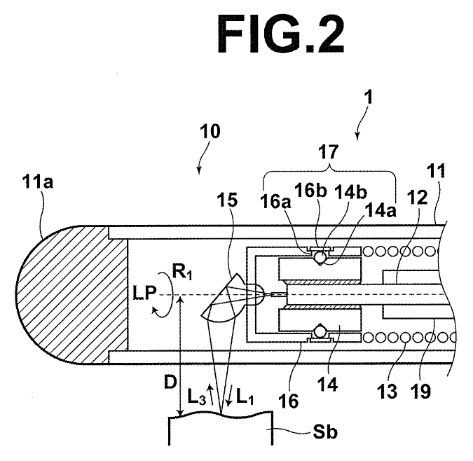

FIG. 2 illustrates an embodiment of the distal end portion 10 of the OCT optical probe 1. The distal end portion 10 of the OCT optical probe 1 includes: a substantially cylindrical flexible sheath 11; the optical fiber 12 contained in and extending along the longitudinal direction of the sheath 11; a rotatably-supporting portion 14 integrally fixed to the optical fiber 12 in the vicinity of the distal end of the optical fiber 12; a distal optical system 15 for collecting and directing the light emitted from the distal end of the optical fiber 12 to the subject; a holding portion 16 for holding the distal optical system 15 such that the distal optical system 15 is rotatably supported by the rotatably-supporting portion 14; and a flexible shaft 13 covering the optical fiber 12. The distal end of the sheath 11 is closed with a cap 11a.

The optical fiber 12 is inserted into and fixed to the rotatably-supporting portion 14 with an adhesive. The measurement light L1 emitted from the distal end of the optical fiber 12 enters the distal optical system 15, and reflected light L3 enters the distal end of the optical fiber 12 via the distal optical system 15.

Preventing unnecessary reflected light from the optical fiber 12 and distal optical system 15 can advantageously improve sensitivity to the interference signal. For example, the amount of reflected light at the distal end of the optical fiber 12 can be reduced by cutting the distal end of the optical fiber 12 obliquely. Further, the amount of reflected light re-entering the optical fiber 12 can be reduced by providing a curved light input surface at the distal optical system 15. In addition, a cover glass, which has a refractive index matched with the optical fiber 12 and has a distal end face that is flat and perpendicular to an optical axis LP, may be provided between the distal end of the optical fiber 12 and the light entrance surface of the distal optical system 15, and the proximal end of the cover glass may be closely bonded to the distal end of the optical fiber 12 with an adhesive. That is, according to this method, reflection at the distal end the optical fiber 12 can be reduced by refraction matching and re-entrance of the reflected light at the distal end of the cover glass into the optical fiber 12 can be reduced by spread of the measurement light L1, thereby reducing the amount of the light re-entering into the optical fiber 12. The distal end of the cover glass may be provided with an AR coating. This method is applicable to either of the cases where the distal end of the optical fiber 12 is flat, and the distal end of the optical fiber 12 is obliquely cut. It should be noted that the structure for reducing the amount of the reflected light usable in the invention is not limited to those described above.

The distal optical system 15 has a substantially spherical shape. The distal optical system 15 deflects the measurement light L1 emitted from the optical fiber 12 and collects and directs the measurement light L1 toward the subject to be measured Sb. The distal optical system 15 also deflects the reflected light L3 from the subject to be measured Sb and collects and directs the reflected light L3 toward the optical fiber 12. The focal length (focal position) of the distal optical system 15 is formed, for example, at a distance D=around 3 mm in the radial direction of the sheath 11 from the optical axis LP of the optical fiber 12. The measurement light L1 emitted from the distal optical system 15 is inclined by an angle of about seven degrees from a direction perpendicular to the optical axis LP. The distal optical system 15 is fixed to the holding portion 16 with an adhesive.

The holding portion 16 is fitted around the rotatably-supporting portion 14 such that a plurality of bearing balls 14b in a groove 14a formed in the outer circumferential surface of the rotatably-supporting portion 14 are respectively positioned in a plurality of holes 16a formed in the inner circumferential surface of the holding portion 16, to form a bearing portion 17. Thus, the holding portion 16 is held rotatably about the optical axis LP relative to the rotatably-supporting portion 14.

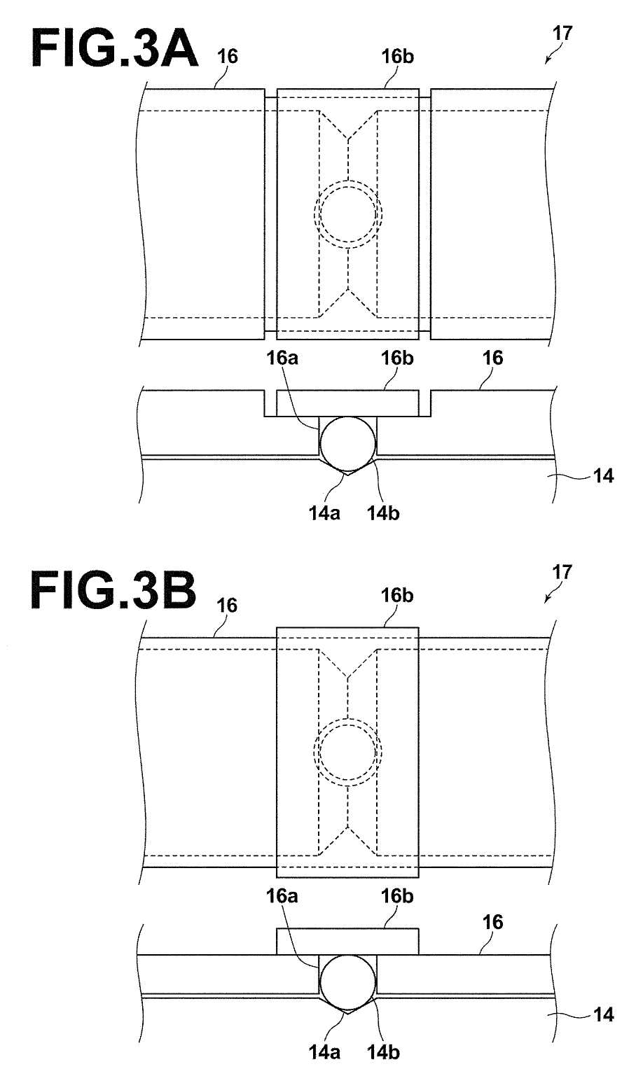

The bearing portion 17 is described in detail. FIG. 3A illustrates a first embodiment of the bearing portion 17 of the OCT optical probe 1, and FIG. 3B illustrates a second embodiment of the bearing portion 17 of the OCT optical probe 1. FIGS. 3A and 3B each shows a side sectional view (at the bottom in the drawing) and a front view (at the top in the drawing) of the bearing portion 17. In the first embodiment, the bearing balls 14b are prevented from falling off by a ring 16b being fitted around the groove formed in the outer circumference of the holding portion 16, as shown in FIG. 3A. The ring 16b may not necessary be completely fixed to the holding portion 16, and may be rotatable within the groove. Further, the ring 16b may have a retainer structure that prevents collision between the adjacent bearing balls 14b. In the second embodiment shown in FIG. 3B, if the diameter of the bearing balls 14b is relatively large with respect to the thickness of the holding portion 16, the bearing balls 14b can be prevented from falling off by fixing the inner circumference of the ring 16b to the outer circumference of holding portion 16 with an adhesive, or the like. If the bearing balls 14b project from the outer circumference surface of the holding portion 16, a groove may be provided in the inner circumference surface of the ring 16b. It should be noted that, in the first and second embodiments, the ring 16b should not hinder the rotation of the bearing balls 14b. Further, the bearing portion 17 may use an oilless bush, or the like, in stead of the bearing balls 14b at the holding portion 16, so that the holding portion 16 slidably rotates about the optical axis LP relative to the rotatably-supporting portion 14.

Referring again to FIG. 2, the flexible shaft 13 is formed by a closed coil spring of a metal wire that is closely wound in a spiral form. The distal end of the flexible shaft 13 is fixed to the holding portion 16, so that the flexible shaft 13 and the holding portion 16 are rotatable about the optical axis LP relative to the rotatably-supporting portion 14. It should be noted that the holding portion 16 may not necessary be fixed to the strictly distal end of the flexible shaft 13, and may be fixed to a portion of the flexible shaft 13 in the vicinity of the distal end thereof. Further, a fiber sheath 19 is provided between the optical fiber 12 and the flexible shaft 13 to reduce rotation of the optical fiber 12 about the optical axis LP due to a frictional force from the rotating flexible shaft 13. In addition, by bonding the fiber sheath 19 to the rotatably-supporting portion 14, durability against frictional wear due to the rotating flexible shaft 13 can be increased. It should be noted that, in stead of providing the fiber sheath 19, the flexible shaft 13 may have a double shaft structure formed by an outer shaft and an inner shaft which are independent from each other.

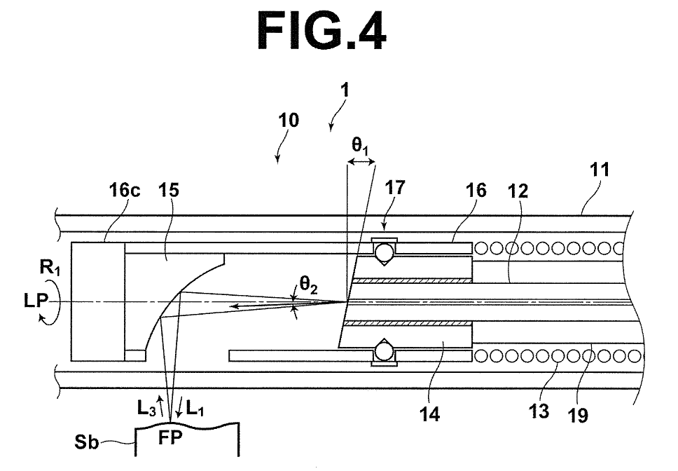

Now, another embodiment of the distal optical system is described. FIG. 4 illustrates the OCT optical probe 1 including a reflecting member. It should be noted that components shown in the drawing which are the same as those in the previous embodiment are designated by the same reference numerals, and explanations thereof are omitted.

In this embodiment, the distal optical system is formed by a reflecting member 15 having a concave surface, and is fixed to the holding portion 16. Although the holding member 16 shown in FIG. 4 is formed by two parts in view of convenience of manufacture, this is not intended to limit the invention. Namely, a cap 16c for holding the reflecting member 15 is fitted on the holding member 16. The concave surface deflects the measurement light L1 emitted from the optical fiber 12 and collects and directs the measurement light L1 toward the subject to be measured Sb. Further, the concave surface deflects the reflected light L3 from the subject to be measured Sb and collects and directs the reflected light L3 toward the optical fiber 12. In this embodiment, the measurement light L1 emitted from the optical fiber 12 and applied to the subject to be measured Sb is reflected only by the concave surface, and therefore, reflection surfaces that generate unnecessary reflected light can be reduced.

Further, in the embodiment shown in FIG. 4, the end face of the rotatably-supporting portion 14 near the reflecting member 15 is polished together with the distal end of the optical fiber 12 so that the distal end face of the optical fiber 12 has a predetermined inclination angle θ1 with respect to the plane perpendicular to the optical axis LP. In this manner, unnecessary reflected light at the distal end of the optical fiber 12 can be reduced, as described above. The inclination angle θ1 is, for example, seven degrees based on APC (Angled PC) polishing standard, however, this is not intended to limit the invention. Further, polishing the optical fiber 12 together with the rotatably-supporting portion 14 is for convenience of manufacture, and this is not intended to limit the invention. It should be noted that inclining the distal end face of the optical fiber 12 with respect to the plane perpendicular to the optical axis LP is also applicable to the embodiment shown in FIG. 2, in which the substantially spherical distal optical system 15 is employed.

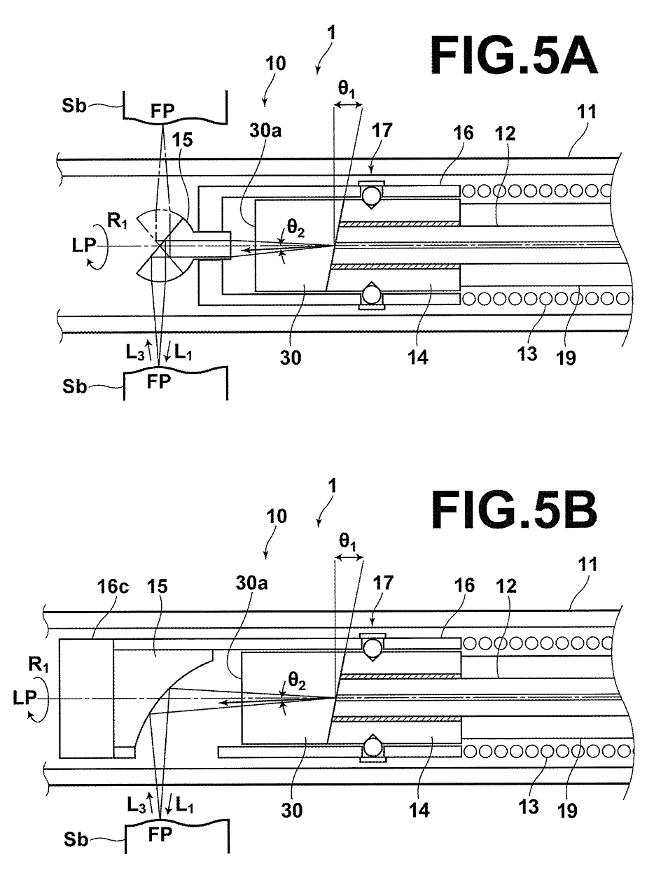

FIGS. 5A and 5B illustrate the OCT optical probe 1 including a cover glass. In the case where the distal end face of the optical fiber 12 has the inclination angle θ1 with respect to the plane perpendicular to the optical axis LP, the direction in which the measurement light L1 is emitted has an emission angle θ2 with respect to the optical axis LP. In general, if the inclination angle θ1 is seven degrees, the emission angle is four degrees. Therefore, as the holding portion 16 rotates, as shown in FIG. 5A, a focal position FP of the measurement light L1 may be shifted in the direction of the optical axis LP between when the measurement light L1 irradiates the upper portion of the subject to be measured Sb in the drawing and when the measurement light L1 irradiates the lower portion of the subject to be measured Sb in the drawing.

As shown in FIGS. 5A and 5B, the cover glass 30 has a refractive index matched with that of the optical fiber 12, and is positioned between the distal end of the optical fiber 12 and the substantially spherical distal optical system 15 or the reflecting member 15. Further, the cover glass 30 is held by the rotatably-supporting portion 14, the proximal end of the cover glass 30 is bonded to the distal end of the optical fiber 12, and the distal end 30a of the cover glass 30 has a flat end face that is perpendicular to the optical axis. It should be noted that the distal end 30a of the cover glass may be provided with an AR coating. By providing the cover glass having the refractive index matched with the optical fiber 12, the emission angle 92 of the measurement light L1 can be reduced from that in a case where the measurement light L1 is guided in the air without using the cover glass 30. Specifically, by providing the cover glass 30, the emission angle θ2 can be reduced to substantially 0 degree.

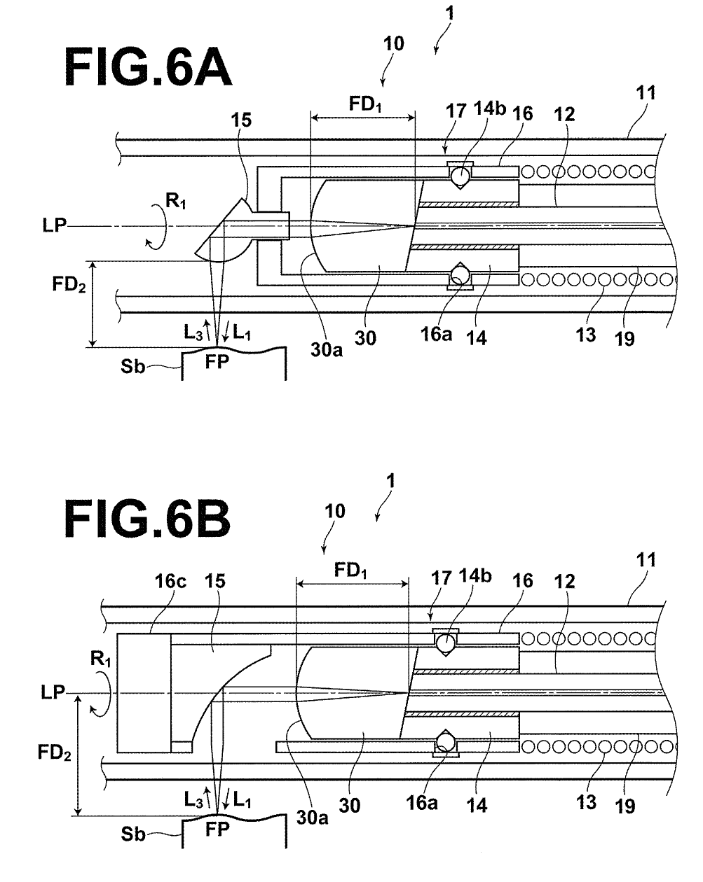

FIGS. 6A and 6B illustrate the OCT optical probe 1 including a cover glass with a convex distal end face. Due to a clearance between the bearing balls 14b and the holes 16a formed in the outer circumference surface of the holding portion 16, the holding portion 16 moves in the direction of the optical axis LP relatively to the rotatably-supporting portion 14. Therefore, a distance between the distal end of the optical fiber 12 and the light entrance surface of the distal optical system 15 or the reflecting member 15 may fluctuate, and this may cause fluctuation of the spot size of the measurement light L1 at the focal position FP. Specifically, the clearance between the bearing balls 14b and the holes 16a is around 100 μm, for example.

In the embodiment shown in FIGS. 6A and 6B, the convex face of the distal end 30a of the cover glass 30 serves to collimate the measurement light L1 emitted from the distal end 30a to be parallel to the optical axis LP. Thus, the spot size of the measurement light L1 at the focal position FP is determined by a ratio between a distance FD1 from the distal end of the optical fiber 12 to the convex surface 30a and a distance FD2 from the distal optical system 15 or reflecting member 15 to the focal position FP, and therefore the spot size at the focal position FP is less susceptible to the fluctuation of the distance from the distal end of the optical fiber 12 to the light entrance surface of the distal optical system 15 or the reflecting member 15. In a case where the cover glass 30 is formed by a lens with distributed refractive index, the distance FD1 from the distal end of the optical fiber 12 to the convex surface 30a can be made shorter than that in a case where a lens with uniform refractive index is used.

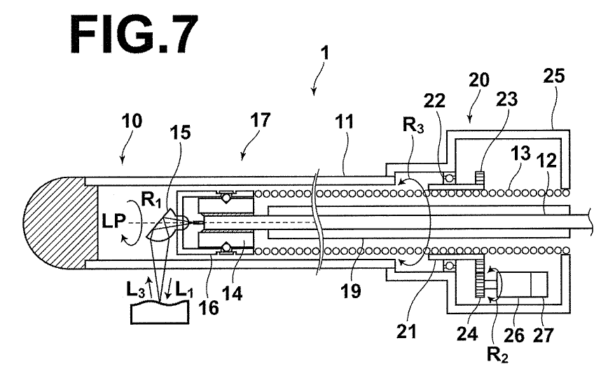

Now, a first embodiment of the OCT optical probe 1 of the invention is described. FIG. 7 illustrates the first embodiment of the OCT optical probe 1.

In the first embodiment, the sheath 11 is fitted in and fixed to a housing 25, and a shaft bearing 22 is disposed in the housing 25. The flexible shaft 13 is fixed to a shaft supporting member 21, and the shaft supporting member 21 is held to be rotatable relative to the housing 25 via the shaft bearing 22. The optical fiber 12 is fixed to the housing 25. A driven gear wheel 23 is fixed to the outer circumference of the shaft supporting member 21, and a driving gear wheel 24 is disposed to mesh with the driven gear wheel 23. The driving gear wheel 24 is fixed to the output shaft of the motor 26, which is disposed in the housing 25. The motor 26 includes an encoder 27 for detecting a rotational angle. A control signal MC fed to the motor 26 and a rotation signal RS fed from the encoder 27 are transmitted via a control cable (not shown). Specifically, the rotation signal RS includes a rotation clock signal RCLK, which is generated for each rotation of the motor 26, and a rotational angle signal Rₚₒₛ.

Now, operation of the first embodiment is described. As the motor 26 rotates in the direction of arrow R2, the shaft supporting member 21 and the flexible shaft 13 fixed to the shaft supporting member 21 rotate, via the driven gear wheel 23 and the driving gear wheel 24, relative to the housing 25 in the direction of arrow R3. This also makes the distal optical system 15, which is fixed to the holding portion 16 at the distal end of the flexible shaft 13, rotate via the bearing portion 17 relatively to the rotatably-supporting portion 14 about the optical axis LP in the direction of arrow R1. Therefore, the OCT optical probe 1 applies the measurement light L1 emitted from the distal optical system 15 to the subject to be measured Sb with moving the measurement light L1 to scan in the direction of arrow R1 about the optical axis LP, i.e., along the circumferential direction of the sheath 11. Specifically, the rotational frequency is around 10-30 Hz, however, this is not intended to limit the invention. If the processing speed of a tomographic image processing unit 150, which will be described later, is high, a higher rotation speed can be used. The rotational frequency may not necessarily be fixed, and may be changed depending on the speed of movement of or the resolution required for the subject to be measured Sb. Specifically, a higher rotation speed maybe used for a subject to be measured Sb that has a high speed of movement or that does not require a high resolution, and a lower rotation speed may be used for a subject to be measured Sb that has a low speed of movement or that requires a high resolution.

Further, the distal optical system 15 can be pivoted about the optical axis LP within a predetermined range of angle by controlling the direction of rotation of the motor 26 according to the control signal MC based on the rotation signal RS. The range of pivot angle can be set to a desirable range based on the shape of the subject to be measured Sb. For example, for a subject to be measured Sb having a cylindrical shape, such as a bronchial tube, the range of pivot angle may be substantially 360 degrees about the longitudinal axis, and for a subject to be measured Sb having a flat shape, such as stomach wall, the range of pivot angle may be around 180 degrees about the longitudinal axis, however, this is not intended to limit the invention. The frequency of pivot is the same as the above-described frequency of rotation. Further, if the frequency of pivot is equal to the natural frequency of the flexible shaft 13, the flexible shaft 13 is resonantly driven, and therefore a driving force can be reduced.

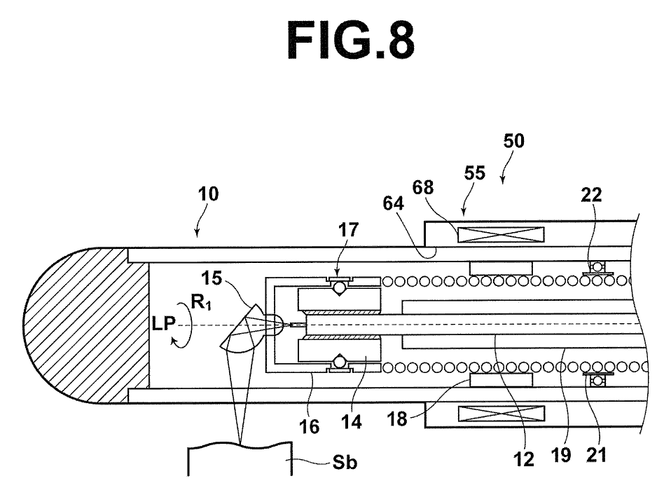

Now, a second embodiment of the OCT optical probe 1 of the invention is described. FIG. 8 illustrates the second embodiment of the invention. Components shown in FIG. 8 that are the same as those of the first embodiment are designated by the same reference numerals, and explanations thereof are omitted. Specifically, features of the second embodiment that are different from the first embodiment are described.

In the second embodiment shown in FIG. 8, a permanent magnet 18 is disposed at the outer circumference of the flexible shaft 13, and an electric magnet 68 is disposed at the outer circumference of the forceps channel 64 of the insert portion 55 of the endoscope 50. Further, a magnetic sensor (not shown) may be disposed at the outer circumference of the permanent magnet 18 for detecting the rotational angle of the optical fiber 12. A control signal MC fed to the electric magnet 68 and a rotation signal RS fed from the magnetic sensor are transmitted via a control cable (not shown). Specifically, the rotation signal RS include a rotation clock signal R_{CLK}, which is generated for each rotation of the flexible shaft 13, and a rotational angle signal Rₚₒₛ.

Now, operation of the second embodiment is described. When the electric magnet 68 is excited, the electric magnet 68 and the permanent magnet 18 interact with each other to establish a relationship of a stator and a rotor of a brushless motor, and thus the flexible shaft 13 rotates in the direction of arrow R3 about the optical axis LP via the permanent magnet 18.

Further, the direction of rotation of the optical fiber 12 may be inverted to make the distal optical system 15 pivot about the optical axis LP within a predetermined range of angle by controlling the order of excitation of the electric magnet 68 according to the control signal MC based on the rotation signal RS.

It should be noted that, in the second embodiment of the invention, the electric magnet 68 may be disposed at the outer circumference of the flexible shaft 13, and the permanent magnet 18 may be disposed at the outer circumference of the forceps channel 64. In this case, the distal end portion 10 is insulated so that the excitation of the electric magnet 68 at the outer circumference of flexible shaft 13 may not exert adverse effect, such as electrical shock, on the human body.

The operation effected by the rotation of the flexible shaft 13 is the same as that in the first embodiment, and explanation thereof is omitted. Further, the pivot angle and the frequency of rotation and pivot are the same as those in the first embodiment, and explanations thereof are omitted.

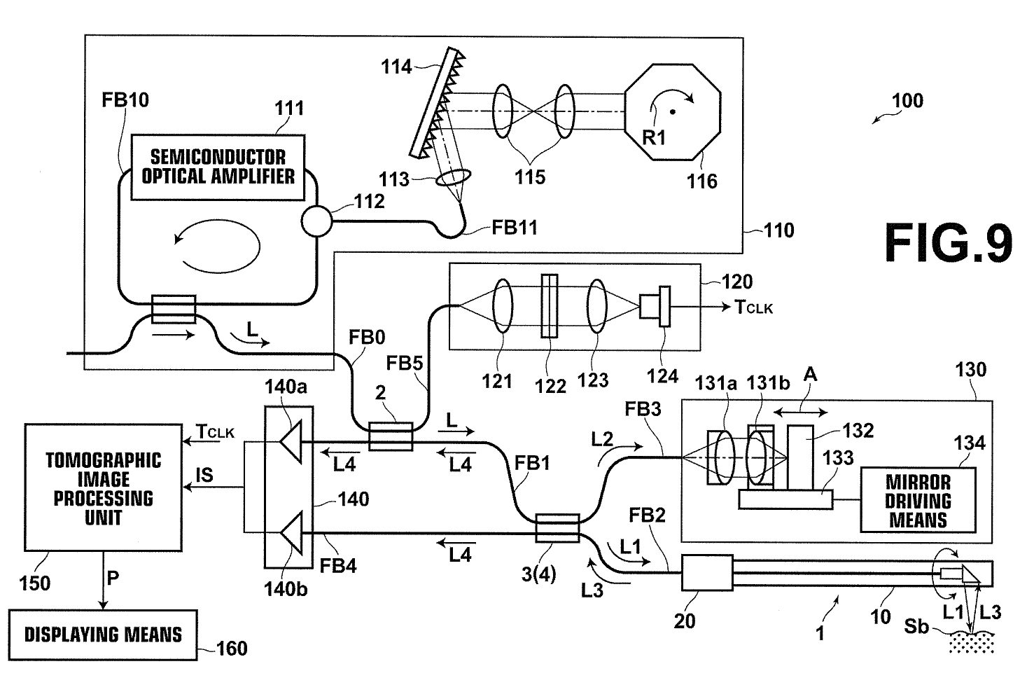

Now, the optical tomography imaging apparatus, to which the OCT optical probe 1 according to the invention is applied, is described. FIG. 9 is a schematic structural diagram of an optical tomography imaging apparatus 100, to which the OCT optical probe 1 of the invention is applied.

The optical tomography imaging apparatus 100 is an optical tomography imaging apparatus using SS-OCT measurement. The optical tomography imaging apparatus 100 includes: a light source unit 110 for emitting laser light L; an optical fiber coupler 2 for dividing the laser light L emitted from the light source unit 110; a period clock generating unit 120 for outputting a period clock signal T_{CLK} from the light divided by the optical fiber coupler 2; a light dividing means 3 for dividing one of light beams divided by the optical fiber coupler 2 into the measurement light L1 and the reference light L2; an optical path length adjusting unit 130 for adjusting the optical path length of the reference light L2 divided by the light dividing means 3; the OCT optical probe 1 for guiding the measurement light L1 divided by the light dividing means 3 to the subject to be measured Sb; a combining means 4 for combining the reference light L2 with the reflected light L3 from the subject to be measured Sb when the measurement light L1 emitted from the OCT optical probe 1 is applied to the subject Sb; an interference light detecting unit 140 for detecting interference light L4 formed between the reflected light L3 and the reference light L2 combined by the combining means 4; a tomographic image processing unit 150 for acquiring a tomographic image P of the subject to be measured Sb by applying frequency analysis to the interference light L4 detected by the interference light detecting unit 140; and a displaying means 160 for displaying the tomographic image P.

The light source unit 110 in this apparatus emits the laser light L with the wavelengths thereof swept in a constant period T0. Specifically, the light source unit 110 includes a semiconductor optical amplifier (semiconductor gain medium) 111 and an optical fiber FB10. The optical fiber FB10 is connected to opposite ends of the semiconductor optical amplifier 111. When a driving current is injected, the semiconductor optical amplifier 111 emits weak light to one end of the optical fiber FB10, and amplifies the light inputted from the other end of the optical fiber FB10. As the driving current is supplied to the semiconductor optical amplifier 111, pulsed laser light L generated by an optical resonator formed by the semiconductor optical amplifier 111 and the optical fiber FB10 is emitted to the optical fiber FB0.

Further, a circulator 112 is coupled to the optical fiber FB10, so that a portion of light guided through the optical fiber FB10 is emitted from the circulator 112 to an optical fiber FB11. The light emitted from the optical fiber FB11 travels through a collimator lens 113, a diffraction optical element 114 and an optical system 115, and is reflected by a rotating polygon mirror 116. The reflected light travels back through the optical system 115, the diffraction optical element 114 and the collimator lens 113, and re-enters the optical fiber FB11.

The rotating polygon mirror 116 rotates at a high speed, such as around 30,000 rpm, in the direction of arrow R1, and the angle of each reflection facet with respect to the optical axis of the optical system 115 varies. Therefore, among the spectral components of the light split by the diffraction optical element 114, only the component of a particular wavelength range returns to the optical fiber FB11. The wavelength of the light returning to the optical fiber FB11 is determined by an angle between the optical axis of the optical system 115 and the reflection facet. Then, the light of the particular wavelength range entering the optical fiber FB11 is inputted from the circulator 112 to the optical fiber FB10. As a result, the laser light L of the particular wavelength range is emitted to the optical fiber FB0.

Therefore, when the rotating polygon mirror 116 rotates at a constant speed in the direction of arrow R1, the wavelength λ of the light re-entering the optical fiber FB11 varies with time in a constant period. As shown in FIG. 10, the light source unit 110 emits the laser light L with the wavelength thereof swept from a minimum sweep wavelength λmin to a maximum sweep wavelength λmax in a constant period T0 (for example, about 50 μsec).

The wavelength-swept laser light L is emitted to the optical fiber FB0, and the laser light L is further inputted to branched optical fibers FB1 and FB5 by the optical fiber coupler 2. The light emitted to the optical fiber FB5 is guided to the period clock generating unit 120.

The period clock generating unit 120 outputs the period clock signal T_{CLK} each time the wavelength of the laser light L emitted from the light source unit 110 is swept for one period. The period clock generating unit 120 includes optical lenses 121 and 123, an optical filter 122 and a photodetector unit 124. The laser light L emitted from the optical fiber FB5 enters the optical filter 122 via the optical lens 121. The laser light L transmitted through the optical filter 122 is then detected by the photodetector unit 124 via the optical lens 123, and the period clock signal T_{CLK} is outputted to the tomographic image processing unit 150.

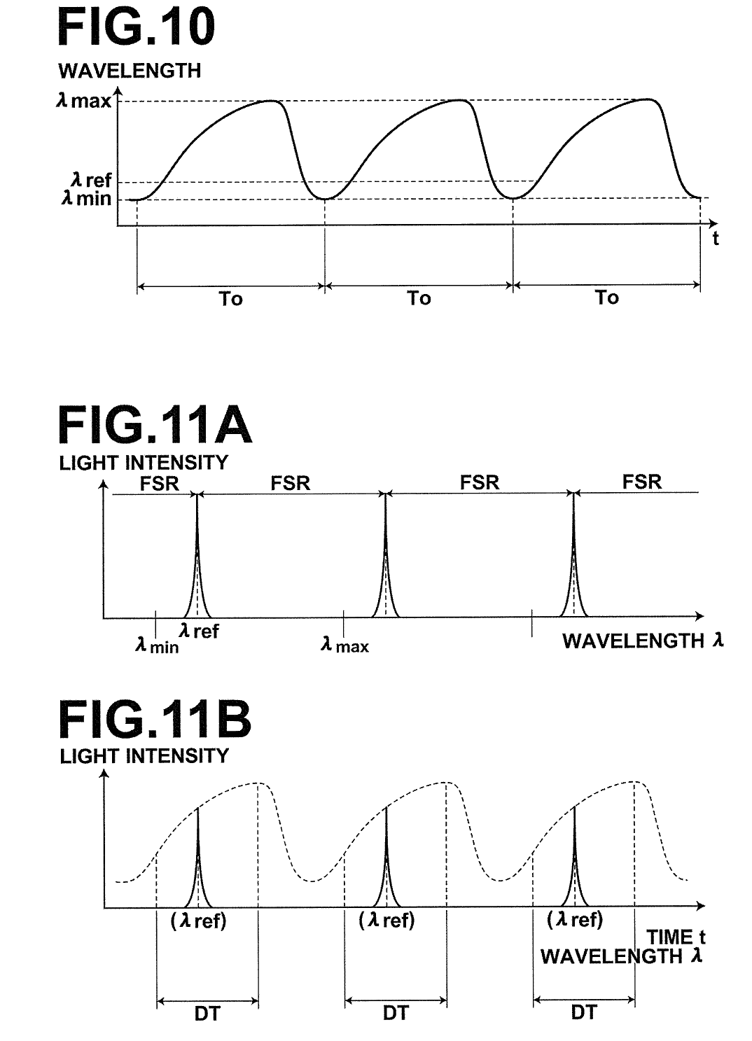

As shown in FIG. 11A, the optical filter 122 transmits only the laser light L having a set wavelength λref, and blocks the light of other wavelength bands. The optical filter 122 has a plurality of transmission wavelengths. The optical filter 122 has a FSR (free spectrum range), which is a light transmission period in which one of the plurality of transmission wavelengths is set within the wavelength band of λmin-λmax. Therefore, only the laser light L having the set wavelength λref within the wavelength band of λmin-λmax, within which the wavelength of the laser light L emitted from the light source unit 110 is swept, is transmitted, and the laser light L of other wavelength bands is blocked.

As shown in FIG. 11B, the period clock signal T_{CLK} is outputted when the wavelength of the laser light L with the periodically swept wavelength emitted from the light source unit 110 is the set wavelength λref. By generating and outputting the period clock signal T_{CLK} using the laser light L actually emitted from the light source unit 110 in this manner, an interference signal IS of the wavelength band of the constant period T0 (see FIG. 10) can be acquired based on the set wavelength λref, even if the time taken for the intensity of the laser light L emitted from the light source unit 110 to reach a predetermined light intensity from the start of sweeping of the wavelength varies for each period. Thus, the period clock signal T_{CLK} can be outputted at timing when the interference signal IS of the wavelength band assumed for the tomographic image processing unit 150 should be acquired, thereby minimizing degradation of resolution.

The light dividing means 3 is formed, for example, by a 2×2 optical fiber coupler, and divides the laser light L guided from the light source unit 110 via the optical fiber FB1 into the measurement light L1 and the reference light L2. Two optical fibers FB2 and FB3 are optically connected to the light dividing means 3, so that the measurement light L1 is guided through the optical fiber FB2 and the reference light L2 is guided through the optical fiber FB3. It should be noted that the light dividing means 3 in this embodiment also serves as the combining means 4.

The optical fiber FB2 is optically connected to the OCT optical probe 1, so that the measurement light L1 is guided to the OCT optical probe 1. The OCT optical probe 1 applies the measurement light L1 emitted from the distal end portion 10 to the subject to be measured Sb, and the reflected light L3 is guided by the optical fiber FB2 through the OCT optical probe 1.

The optical path length adjusting unit 130 is disposed at the side of the optical fiber FB3 from which the reference light L2 is emitted. The optical path length adjusting unit 130 changes the optical path length of the reference light L2 to adjust the position at which acquisition of the tomographic image is started. The optical path length adjusting unit 130 includes: a reflection mirror 132 for reflecting the reference light L2 emitted from the optical fiber FB3; a first optical lens 131a disposed between the reflection mirror 132 and the optical fiber FB3; and a second optical lens 131b disposed between the first optical lens 131a and the reflection mirror 132.

The first optical lens 131a serves to collimate the reference light L2 emitted from the optical fiber FB3 and to collect the reference light L2 reflected from the reflection mirror 132 onto the optical fiber FB3.

The second optical lens 131b serves to collect the reference light L2 collimated by the first optical lens 131a onto the reflection mirror 132 and to collimate the reference light L2 reflected from the reflection mirror 132.

That is, the reference light L2 emitted from the optical fiber FB3 is collimated by the first optical lens 131a, and then is collected by the second optical lens 131b onto the reflection mirror 132. Thereafter, the reference light L2 reflected from the reflection mirror 132 is collimated by the second optical lens 131b, and then is collected by the first optical lens 131a onto the optical fiber FB3.

The optical path length adjusting unit 130 further includes: a base 133 on which the second optical lens 131b and the reflection mirror 132 are fixed; and a mirror moving means 134 for moving the base 133 along the optical axis of the first optical lens 131a. The optical path length of the reference light L2 can be changed by moving the base 133 in the direction of arrow A.

The combining means 4 is formed by a 2×2 optical fiber coupler, as described above. The combining means 4 is adapted to combine the reference light L2 having the optical path length adjusted by the optical path length adjusting unit 130 with the reflected light L3 from the subject to be measured Sb, and emit the combined light to the interference light detecting unit 140 via the optical fiber FB4.

The interference light detecting unit 140 detects the interference light L4 between the reflected light L3 and the reference light L2 combined by the combining means 4, and outputs the interference signal IS. It should be noted that, in this apparatus, the interference light L4 is divided into two parts by the light dividing means 3 and these parts are guided to the photodetectors 140a and 140b to be calculated, so that balanced detection is carried out. The interference signal IS is outputted to the tomographic image processing unit 150.

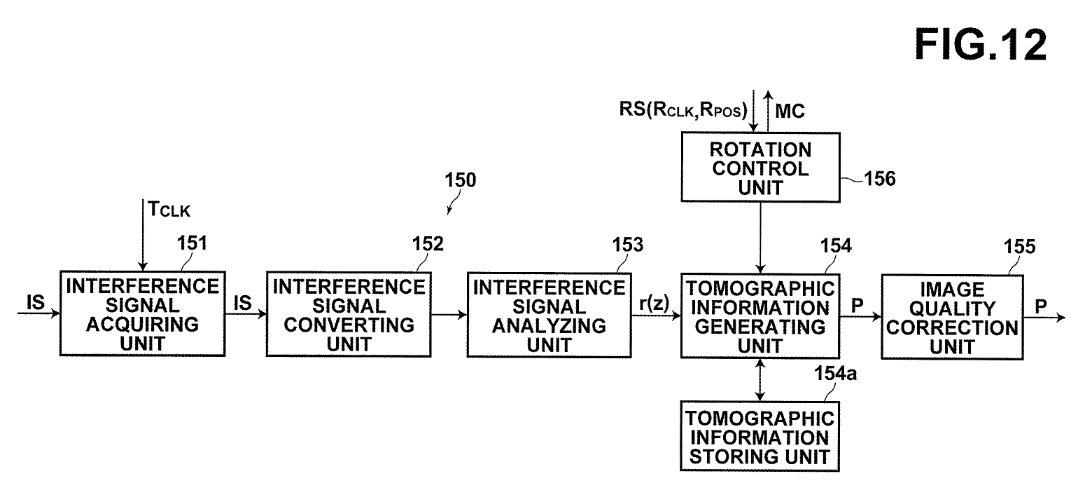

FIG. 12 is a schematic structural diagram of the tomographic image processing unit 150. The tomographic image processing unit 150 is implemented by executing a tomographic imaging program, which is installed in an auxiliary storage device of a computer (for example, personal computer), on the computer. The tomographic image processing unit 150 includes an interference signal acquiring unit 151, an interference signal converting unit 152, an interference signal analyzing unit 153, a tomographic information generating unit 154, an image quality correction unit 155 and a rotation control unit 156.

The interference signal acquiring unit 151 acquires the interference signal IS for one period, which is detected by the interference light detecting unit 140, based on the period clock signal T_{CLK} outputted from the period clock generating unit 120. The interference signal acquiring unit 151 acquires the interference signal IS of a wavelength band DT (see FIG. 11B) spanning between points before and after the output timing of the period clock signal T_{CLK}. It should be noted that the output timing of the period clock signal T_{CLK} may be set immediately after the start of the wavelength sweeping or immediately before the end of the wavelength sweeping, as long as it is within the wavelength band to be swept, so that the interference signal acquiring unit 151 can acquire the interference signal IS for one period based on the output timing of the period clock signal T_{CLK}.

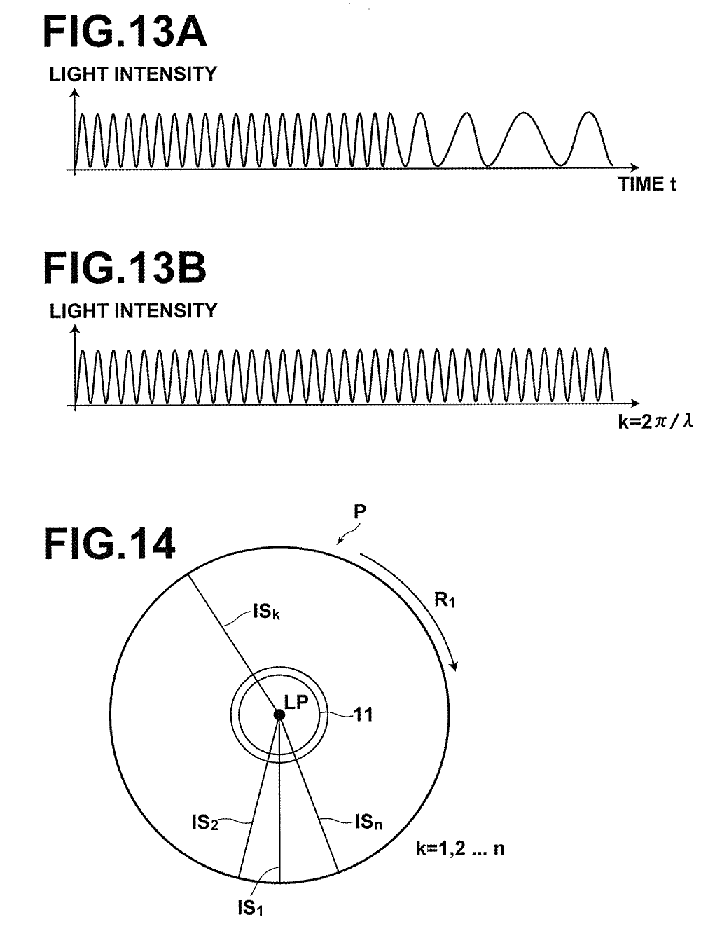

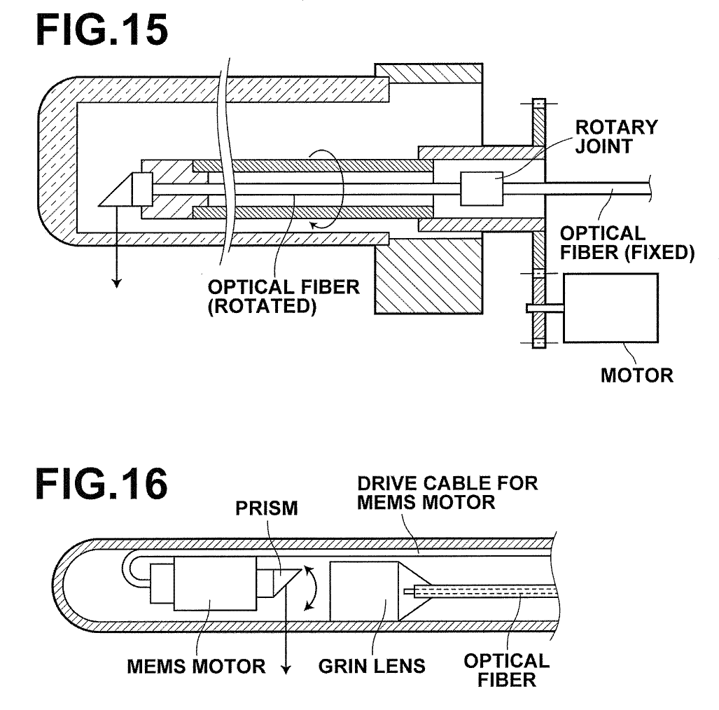

The interference signal converting unit 152 rearranges the interference signal IS acquired by the interference signal acquiring unit 151 in equal intervals along the wavenumber k (=2π/λ) axis. FIG. 13A illustrates the interference signal IS to be inputted to the interference signal acquiring unit 151. FIG. 13B illustrates the rearranged interference signal IS. Specifically, the interference signal converting unit 152 is provided in advance with a time-wavelength sweep characteristics data table or function of the light source unit 110, and uses this time-wavelength sweep characteristics data table to rearrange the interference signal IS in equal intervals along the wavenumber k axis. This allows acquisition of highly accurate tomographic information by using a spectral analysis technique that assumes that the data is arranged in equal intervals in a frequency space, such as the Fourier transformation or processing using the maximum entropy method, to calculate the tomographic information from the interference signal IS. Details of this signal conversion technique is disclosed in U.S. Pat. No. 5,956,355.

The interference signal analyzing unit 153 acquires the tomographic information r(z) by applying a known spectral analysis technique, such as the Fourier transformation, the maximum entropy method, or the Yule-Walker method, to the interference signal IS converted by the interference signal converting unit 152.

The rotation control unit 156 outputs the control signal MC to the motor 26 or the electric magnet 68, and receives the rotation signal RS inputted from the encoder 27 or the magnetic sensor. As described above, the rotational position signal RS includes the rotation clock signal R_{CLK}, which is generated for each rotation of the motor 26 or the flexible shaft 13, and the rotational angle signal Rₚₒₛ.

The tomographic information generating unit 154 acquires the tomographic information r(z), which corresponds to scanning by the distal end portion 10 of the OCT optical probe 1 in the radial direction (in the direction of arrow R1 in the drawing), for one period (one line) acquired by the interference signal analyzing unit 153, and generates a tomographic image P as shown in FIG. 14. The tomographic information generating unit 154 stores the tomographic information r(z) for one line, which is sequentially acquired, in a tomographic information storing unit 154a.

The tomographic information generating unit 154 can generate the tomographic image P by reading the tomographic information r(z) for n lines at a time from the tomographic information storing unit 154a based on the rotation clock signal RCLK inputted to the rotation control unit 156.

Alternatively, the tomographic information generating unit 154 can generate the tomographic image P by sequentially reading the tomographic information r(z) from the tomographic information storing unit 154a based on the rotational angle signal Rₚₒₛ inputted to the rotation control unit 156.

The image quality correction unit 155 applies correction, such as sharpness correction and smoothness correction, to the tomographic image P generated by the tomographic information generating unit 154.

The displaying means 160 displays the tomographic image P, which has been subjected to the correction, such as sharpness correction and smoothness correction, applied by the image quality correction unit 155.

As described above, in the OCT optical probe 1 of the invention and the optical tomography imaging apparatus 100 employing the OCT optical probe 1, no rotary joint is provided, and the light emitted from the distal end of the optical fiber 12 directly enters the distal optical system 15. Therefore, the problem of degradation of measurement accuracy due to the optical insertion loss and optical reflection loss at the rotary joint can be eliminated inexpensively and safely.

Also, in the optical tomography imaging apparatus 100 according to the invention, to which the above-described OCT probe 1 of the invention is applied, the problem of degradation of measurement accuracy due to the optical insertion loss and optical reflection loss at the rotary joint can be eliminated inexpensively and safely.

Although the optical tomography imaging apparatus, to which the OCT optical probe 1 of the invention is applied, has been described as an SS-OCT apparatus in the above embodiment by way of example, the OCT optical probe 1 of the invention is also applicable to SD-OCT and TD-OCT apparatuses.

In the OCT optical probe of the invention, the distal optical system is rotated by the flexible shaft via the holding portion relative to the rotatably-supporting portion that is integrally fixed to the distal end portion of the optical fiber. Therefore, the light emitted from the light source unit is guided through the optical fiber and directly enters the distal optical system from the distal end of optical fiber.

Thus, it is not necessary to provide a rotary joint between the distal end portion and the proximal end portion of the OCT optical probe, and therefore the problem of optical insertion loss and optical reflection loss at the rotary joint can be avoided. Further, since no driving means, such as an MEMS motor, is provided in the vicinity of the distal end, problems such as increase of the outer diameter of the OCT optical probe and an adverse effect exerted on image acquisition by a drive cable for the MEMS motor can be avoided.

Using the OCT probe according to the invention, the problem of degradation of measurement accuracy due to the optical insertion loss and optical reflection loss at the rotary joint can be eliminated inexpensively and safely.

Also, in the optical tomography imaging apparatus according to the invention, to which the above-described OCT probe according to the invention is applied, the problem of degradation of measurement accuracy due to the optical insertion loss and optical reflection loss at the rotary joint can be eliminated inexpensively and safely.

What is claimed is:

1. An OCT optical probe comprising:
a substantially cylindrical sheath to be inserted into a subject, the sheath having an internal space;
an optical fiber disposed in the internal space of the sheath along the longitudinal direction of the sheath;
a rotatably-supporting portion integrally fixed to the optical fiber in the vicinity of a distal end of the optical fiber;
a distal optical system to deflect light emitted from the distal end of the optical fiber toward the subject;
a holding portion to hold the distal optical system such that the distal optical system is rotatably supported by the rotatably-supporting portion; and
a flexible shaft covering the optical fiber in the internal space of the sheath, wherein the holding portion is fixed to a distal end of the flexible shaft.

2. The OCT optical probe as claimed in claim 1, wherein the rotatably-supporting portion comprises a bearing portion to rotatably support the holding portion.

3. The OCT optical probe as claimed in claim 1 further comprising a fiber sheath disposed between the optical fiber and the flexible shaft, the fiber sheath covering the optical fiber.

4. The OCT optical probe as claimed in claim 1, wherein the distal end of the optical fiber comprises an end face inclined by a predetermined angle with respect to a plane perpendicular to an optical axis of the optical fiber.

5. The OCT optical probe as claimed in claim 1 further comprising a cover glass, a proximal end of the cover glass closely contacting the distal end of the optical fiber, and a distal end the cover glass comprising a flat end face perpendicular to the optical axis.

6. The OCT optical probe as claimed in claim 1 further comprising a cover glass, a proximal end of the cover glass closely contacting the distal end of the optical fiber, and a distal end of the cover glass comprising a convex end face adapted to collimate the light emitted from the distal end of the cover glass to be parallel to the optical axis.

7. An optical tomography imaging apparatus comprising:
a light source unit to emit light;
a light dividing unit to divide the light emitted from the light source unit into measurement light and reference light;
an irradiation optical system to irradiate a subject to be measured with the measurement light;
a combining unit to combine the reference light with reflected light of the measurement light reflected from the subject to be measured when the measurement light is applied to the subject;
an interference light detecting unit to detect interference light formed between the combined reflected light and reference light; and
a tomographic image processing unit to detect reflection intensity at a plurality of depth-wise positions in the subject to be measured based on frequency and intensity of the detected interference light, and to acquire a tomographic image of the subject to be measured based on the intensity of the reflected light at each of the depth-wise positions,
wherein the irradiation optical system comprises the OCT optical probe as claimed in claim 1.
